SEQUENCE LISTING (30 sequences)

SEQ ID NO: 1 - protein, 25 aa, Oryctolagus cuniculus
DGAIDPYFKIWGPGTLVTVSSGQPK

SEQ ID NO: 2 - protein, 14 aa, Oryctolagus cuniculus
SGQPPKLLIYQASK

SEQ ID NO: 3 - protein, 16 aa, Oryctolagus cuniculus
LLIYQASKVTSGVPSR

SEQ ID NO: 4 - protein, 22 aa, Oryctolagus cuniculus
SGQPPKLLIYQASKVTSGVPSR

SEQ ID NO: 5 - protein, 32 aa, Oryctolagus cuniculus
QVLTQTPSPVSAALGGTVTINCQSSQSVAGNR

SEQ ID NO: 6 - protein, 22 aa, Oryctolagus cuniculus
TSTTVDLKMTSPTTEDTATYFC

SEQ ID NO: 7 - protein, 25 aa, Oryctolagus cuniculus
LLIYQASKVTSGVPSRFSGSGSGTQ

SEQ ID NO: 8 - protein, 8 aa, Oryctolagus cuniculus
KIWGPGTL

SEQ ID NO: 9 - protein, 22 aa, Oryctolagus cuniculus
IDMTQTPSPVSAAVGDTVTISC

SEQ ID NO: 10 - protein, 12 aa, Oryctolagus cuniculus
QTPGKGLELIAC

SEQ ID NO: 11 - protein, 17 aa, Oryctolagus cuniculus
IDISGPYTYYASWAKGR

SEQ ID NO: 12 - protein, 17 aa, Oryctolagus cuniculus
QSLEESGGDLVKPGASL

SEQ ID NO: 13 - protein, 5 aa, Oryctolagus cuniculus
AKGRF

SEQ ID NO: 14 - protein, 11 aa, Oryctolagus cuniculus
QSSQSVYKNNR

SEQ ID NO: 15 - protein, 33 aa, Oryctolagus cuniculus
IDMTQTPSPVSAAVGDTVTISCQSSQSVYKNNR

SEQ ID NO: 16 - protein, 20 aa, Oryctolagus cuniculus
WAFGGGTEVVVKGDPVAPTV

SEQ ID NO: 17 - protein, 14 aa, Oryctolagus cuniculus
ISCQSSQSVYKNNR

SEQ ID NO: 18 - protein, 14 aa, Oryctolagus cuniculus
LAWYQQKPGKPPKL

SEQ ID NO: 19 - protein, 15 aa, Oryctolagus cuniculus
ISCQSSQSVYKNNRL

SEQ ID NO: 20 - protein, 13 aa, Oryctolagus cuniculus
AWYQQKPGKPPKL

SEQ ID NO: 21 - protein, 16 aa, Oryctolagus cuniculus
ISCQSSQSVYKNNRLA

SEQ ID NO: 22 - protein, 12 aa, Oryctolagus cuniculus
WYQQKPGKPPKL

SEQ ID NO: 23 - protein, 17 aa, Oryctolagus cuniculus
ISCQSSQSVYKNNRLAW

SEQ ID NO: 24 - protein, 13 aa, Oryctolagus cuniculus
YQQKPGKPPKLLI

SEQ ID NO: 25 - protein, 11 aa, Oryctolagus cuniculus
YQQKPGKPPKL

SEQ ID NO: 26 - protein, 13 aa, Oryctolagus cuniculus
LAWYQQKPGKPPK

SEQ ID NO: 27 - protein, 213 aa, Oryctolagus cuniculus
QVLTQTPSPVSAALGGTVTINCQSSQSVAGNRWAAWYQQKSGQPPKLLIYQASKVTSGVPSRFSGSGSGTQFTLTISDLECDDAAIYYCAGGYSGEFWAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 28 - protein, 213 aa, Oryctolagus cuniculus
IDMTQTPSPVSAAVGDTVTISCQSSQSVYKNNRLAWYQQKPGQPPKLLIYLASTLASGVPSRFKGSGSGTQFTLTISEVQCDDAATYYCQAYYDGYIWAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 29 - protein, 441 aa, Oryctolagus cuniculus
QSVEESGGRLVTPGTPLTLTCTVSGFSLSSYAMGWVRQAPGKGLEYIGIIDANDYIFYASWAKGRFTISKTSTTVDLKMTSPTTEDTATYFCARYSRDGAIDPYFKIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 30 - protein, 443 aa, Oryctolagus cuniculus
QSLEESGGDLVKPGASLTLTCKASGFSFSSGYDICWVRQTPGKGLELIACIDISGPYTYYASWAKGRFTISKTSSTTVTLQLTSLTAADTATYFCAKTDPTISSSYFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

(19) United States
(12) Patent Application Publication
(10) Pub. No.: US 2024/0280588 A1
(43) Pub. Date: Aug. 22, 2024

(54) SYSTEMS AND METHODS FOR ANTIBODY CHAIN PAIRING

(71) Applicant: Rapid Novor Inc., Kitchener (CA)

(72) Inventors: Thierry LE BIHAN, Guelph, Ontario (CA); Teresa NUNEZ DE VILLAVICENCIO DIAZ, Saint-Colomban, Québec (CA); Mariya LIYASOVA, Kitchener, Ontario (CA); Chenyu YAO, Waterloo, Ontario (CA); Qixin LIU, Waterloo, Ontario (CA); Chelsea REITZEL, Kitchener, Ontario (CA); Jin DUAN, Kitchener, Ontario (CA); Paul TAYLOR, Waterloo, Ontario (CA); Bin MA, Waterloo, Ontario (CA)

(21) Appl. No.: 18/681,198

(22) PCT Filed: Aug. 5, 2022
(86) PCT No.: PCT/CA2022/051194

§ 371 Date: Feb. 5, 2024

Related U.S. Application Data
(60) Provisional application No. 63/260,027, filed on Aug. 6, 2021.

(51) Int. Cl. G01N 33/68; C07K 1/20; C07K 1/36; C12Q 1/37

(52) U.S. Cl. CPC G01N 33/6857; C07K 1/20; C07K 1/36; C12Q 1/37; G01N 33/6848

(57) ABSTRACT

The present application relates to methods for determining antibody heavy and light chain pairing in an antibody and/or antibody fragment mixture. The methods include a step of submitting the mixture to a separation step under non-reducing condition to obtain isolated antibodies or antibody fragments, or of contacting the mixture with a protein cross-linking agent. The isolated or cross-linked antibodies and/or antibody fragments are then digested using one or more proteases to generate antibody or antibody fragment peptides, which are then analyzed for example by mass spectrometry (MS) to determining heavy chain and light chain pairing of the antibody or antibody fragment in the mixture.

### CROSS REFERENCE TO RELATED APPLICATIONS

The present application claims the benefits of U.S. provisional application Ser. No. 63/260,027, filed on Aug. 6, 2021, which is incorporated herein by reference in its entirety.

### TECHNICAL FIELD

The present disclosure generally relates to the field of immunology, and more specifically to antibody analysis and assessment of the antibody repertoire.

### BACKGROUND ART

Antibodies are one of the main effectors of the adaptive immune system. Their ability to bind an antigen has been harnessed in order to generate several tools for diagnostic, research and clinical applications. Antibodies are among the fastest developing biomolecules in clinical trials with a worldwide market estimated at 130.9 billion for 2020 and estimated to grow to 223.7 billion by the end of 2025 (https://www.marketdataforecast.com/market-reports/antibodies-market).

Antibodies are classified as glycoproteins. Antibodies are composed of four polypeptide chains: two identical copies of both a heavy (H, 55 kDa), and a light chain (L, 25 kDa), held together by a disulfide bridge. The basic profile is similar to a “Y” shape. Each polypeptide chain has a constant region, which is conserved across antibodies, and a variable region, which is specific to each antibody thus mainly responsible for conferring affinity to an antigen.

From an application point of view, antibodies can be divided into two main categories: monoclonal antibodies (mAbs), which are produced by a single type of plasma cell, and have the same heavy and light chain sequence and bind to a unique epitope on a given antigen. On the other hand, polyclonal antibodies (pAb) consist of a pool of different antibodies from various B cells recognizing both similar and different parts of the same antigens; those regions are named epitopes.

Antibodies are secreted by plasma cells, which are differentiated mature B lymphocytes (Pioli 2019). Plasma cells produce multiple antibodies in response to an antigen. In 1975, hybridoma technology was developed to immortalize individual plasma cells (Packer 2021). The antibodies produced by a population of homogeneous immortalized plasma cell, denoted as a clone, are called monoclonal antibodies (mAbs). In contrast, antibodies produced from plasma cells in an animal are called polyclonal antibodies (pAbs). Monoclonal antibodies have the same heavy (H) and light (L) chain sequence and bind to a unique epitope on a given antigen. On the other hand, pAbs recognize different parts or epitopes of the same antigen and are a mixture of antibodies having different sequences.

Monoclonal antibodies are developed and used for several biological applications, diagnostics and clinicals use such as treatments for autoimmune disease, cancer and infectious diseases to name a few. However, mAb therapeutics are simplistic versions of the much more complex native immune response, which involves several different antibodies targeting different epitopes (Wang et al 2013).

On the other hand, pAb treatments are available for specific cases such as rabies. Treatments are based on either the use of human rabies immunoglobulin (HRIG), or a cheaper and safe horse version (ERIG). Both are obtained from pooled sera of human donors or horses vaccinated against rabies. Other issues associated with HRIG usage include potential health risks, batch-to-batch variability, and limited global supply in certain regions of the world or under of an unexpected mass exposure. Bakker et al. showed that recapitulation of a pAb mixture by combining a few different monoclonal antibodies targeting different epitopes of the rabies virus glycoproteins could be efficient against different street rabies viruses and against escape mutants of the virus (Bakker, 2005).

Choosing to generate or work with mAbs or pAbs is dictated by different factors, including application type, production cost and time, and technical expertise. Both mAbs and pAbs have different advantages and disadvantages. Polyclonal antibodies can be generated more rapidly than mAbs with less demanding technical skills as all that is required is animal being inoculated with a target antigen and adjuvant. In addition, pAbs are heterogenous in nature, which ensures they can recognize a given epitope under different conformations or with small changes pAbs are also more flexible in term of buffer use and epitope changes. Furthermore, pAb generated from different animals will exhibit variation in their affinity and drastic variation within an animal can be expected.

Unlike pAbs, mAbs are homogeneous, and therefore have low batch-to-batch variability, but their high specificity can limit their use in some cases; for instance, a small change in the structure of the epitope can drastically affect antibody-antigen affinity. Moreover, because mAbs are generated from identical immortalized B-cells, often by fusing a B cell and a myeloma cell, their production can be sustained in vitro with a given specificity.

However, hybridoma cells can be subjected to gene loss, gene mutation, additional chains found in hybridoma and cell line drift. This later problem identified in mAbs can be overcome using recombinant antibodies (rAbs). For recombinant antibody rAbs, the antibody sequence has to be determined in order to synthesize the immunoglobulin (Ig) L and H chain genes and generate expression constructs. The constructs are then transfected into a high yield cell such as CHO or HEK293.

A similar strategy could be applied to pAbs. Being able to sequence several antibodies from a polyclonal mixture, at least some of its main dominant forms, to generate recombinants to produce a simpler complex mixture such as a recombinant oligoclonal mixture is therefore an attractive solution, which combines advantages from both the mAbs and rAbs side (removal of batch-to-batch variability, circumvention of loss of hybridoma), and pAbs (a response closer to that of the natural immune system). To date, very few efforts have been attempted to sequence pAbs.

In an approach developed by Cheung et al., antibodies were first enriched from an immunized animal and analyzed using a standard proteomics mass spectrometry (MS)-based approach searched against a reference database created by Next Generation Sequencing (NGS) of the B cell Ig repertoire of the immunized animal. Pairing of the H and L chains was performed by exploring all possible combinations of H and L chains. A relatively similar approach was proposed by Wine et al. (2013).

Guthals et al 2016, provided an example of sequencing a mixture derived from a polyclonal mixture purified by glycoprotein B antigen affinity from a cytomegalovirus-exposed individual; they managed to sequence several H and L chains and concentrate their efforts on the top four LC and seven HC. They had to generate all possible pairwise combinations expressed in mammalian cells to validate antigen binding. Like previous attempts, the main issue they encountered was that the pairing was blindly explored through a combinatorial approach; such an approach of H and L pairing is feasible for a low number of antibodies but is rather impossible or too demanding for large datasets as it is the multiplication product of the number of identified L chains by the number of identified H chains.

The importance of proper pairing has been discussed in different works. Czerwinski et al (1998) highlighted the significance of proper pairing by screening a library of chimeric antibody antigen-binding domains (Fab). Brandon et al (2013) addressed the H/L pairing by performing single B-cell sequencing. Jared Shaw (2020), proposed a direct chain pairing using top down and middle down mass spectrometry approaches; their proposed approaches rely on the complete knowledge of the different sequences to identify within a pair, and can be performed on intact fragments only.

Most of the proposed strategies used to identify an antibody or several antibodies from a large population are conducted either through proteomics or transcriptomics; the sequencing strategy is performed on samples where H and L chain are under reduced separate conditions (i.e. disulfide bonds disrupted and the molecules being fragmented), and consequently the H/L pairing information is lost during the sequencing procedure. One of the main strategies often used to identify the proper H/L pairs simply consist of generating all permutations by expressing all possible assemblies, and performing a functionality assay, which can be a tedious task for complex samples.

There is thus a need for novel approaches to identify light and heavy chain pairs in complex antibody mixtures such as in polyclonal antibody mixtures, or at least to reduce the number of possible valid pairing to consider.

The present description refers to a number of documents, the content of which is herein incorporated by reference in their entirety.

### SUMMARY

The present disclosure provides the following items 1 to 32:
1. A method for determining heavy and light chain pairing of an antibody or an antibody fragment in an antibody and/or antibody fragment mixture, the method comprising (a) submitting the mixture to a separation step under non-reducing condition to obtain fractions of isolated antibodies or antibody fragments; (b) digesting the isolated antibodies or antibody fragments in the fractions to obtain antibody or antibody fragment peptides; (c) analyzing the antibody or antibody fragment peptides; and (d) determining heavy chain and light chain pairing of the antibody or antibody fragments based on the analysis.
2. A method for determining heavy and light chain pairing of an antibody or an antibody fragment in an antibody and/or antibody fragment mixture, the method comprising (i) contacting the mixture with a cross-linking agent to obtain cross-linked antibodies or antibody fragments; (ii) digesting the cross-linked antibodies or antibody fragments to obtain crosslinked antibody or antibody fragment peptides; (iii) analyzing the crosslinked antibody or antibody fragment peptides; and (iv) determining heavy chain and light chain pairing of the antibody or antibody fragment based on the analysis.
3. The method of item 1, wherein the separation step under non-reducing condition comprises submitting the antibody and/or antibody fragment mixture to a chromatography.
4. The method of item 3, wherein the chromatography is hydrophobic interaction chromatography (HIC).
5. The method of item 1, wherein the separation step under non-reducing condition comprises migrating the antibody and/or antibody fragment mixture on a gel.
6. The method of item 5, wherein the gel is a polyacrylamide or agarose gel.
7. The method of item 5 or 6, wherein the gel is a native gel.
8. The method of item 5 or 6, wherein the gel is a denaturing 2D gel.
9. The method of item 1, wherein the separation step under non-reducing condition comprises submitting the antibody and/or antibody fragment mixture to capillary electrophoresis.
10. The method of item 9, wherein the capillary electrophoresis is imaged capillary isoelectric focusing (iCIEF).
11. The method of any one of items 1 and 3-10, further comprises contacting the antibody and/or antibody fragment mixture with an agent that modifies the charge of the protein molecule in a sequence specific manner.
12. The method of item 11, wherein said agent modifies positively-charged residues into neutral or negative charge groups.
13. The method of item 12, wherein said agent is citraconic anhydride (CA) or sulfo-NHS-acetate (SNA)
14. The method of item 2, wherein the cross-linking agent comprises two N-hydroxysulfosuccinimide (NHS) ester groups.
15. The method of item 14, wherein the cross-linking agent is Bis(sulfosuccinimidyl) suberate (BS3).
16. The method of any one of items 1 to 15, wherein the method further comprises reducing and alkylating the cysteine residues prior to said digesting.
17. The method of any one of items 1 to 16, wherein the antibody and/or antibody fragment mixture is a polyclonal antibody mixture or a mixture of monoclonal antibodies.
18. The method of any one of items 1 to 17, wherein the digesting comprises contacting the antibodies or antibody fragments with at least one protease.
19. The method of item 18, wherein the at least one protease comprises trypsin, chymotrypsin, AspN, GluC, ArgC, LysC, LysN, pepsin or any combination thereof.
20. The method of any one of items 1 to 19, wherein said analyzing comprises quantifying peptide abundance or intensity of peptide signature from the heavy and light chain across the different fractions, and wherein said determining heavy chain and light chain pairing of the antibody or antibody fragments is based on the amount of peptide from the heavy and light across the different fractions.
21. The method of any one of items 1 to 20, further comprising determining the amino acid sequence of at least a portion of the light and heavy chains of the antibodies or antibody fragments present in the mixture.
22. The method of item 21, wherein said portion of the light and heavy chains is the complementary determining region 3 (CDR3).
23. The method of any one of items 1 to 22, wherein the step of analyzing the antibody or antibody fragment peptides comprises submitting the antibody or antibody fragment peptides to mass spectrometry (MS) and sequencing the peptides using tandem MS (MS/MS).
24. The method of item 23, wherein the MS is liquid chromatography-MS (LC-MS) and the MS/MS is liquid chromatography MS/MS (LC-MS/MS).
25. The method of item 23 or 24, wherein the MS/MS spectra are either compared to a database of antibody peptide sequences or to the sequences of at least a portion of the light and heavy chains of the antibodies or antibody fragments present in the mixture as determined according to item 21 or 22.
26. The method of any one of items 1 to 25, wherein the step of analyzing the antibody or antibody fragment peptides comprises performing the quantitative profiling of proteotypic peptides unique to each heavy and light chains, and performing pairing based on co-expression of the proteotypic peptides in the same fraction.
27. The method of any one of items 1 to 25, wherein the step of analyzing the antibody or antibody fragment peptides comprises performing a clustering analysis.
28. The method of item 27, wherein the clustering analysis is principal component analysis (PCA) or cross-correlation analysis.
29. The method of any one of items 1 to 28, further comprising expressing a recombinant antibody or antibody fragment comprising the heavy and light chain pair identified by the method defined in any one of items 1 to 28.
30. The method of claim 29, further comprising assessing the binding of the recombinant antibody or antibody fragment to a target antigen.
31. The method of item 30, wherein assessing the binding of the antibody or antibody fragment comprises performing an immunoassay.
32. The method of item 31, wherein the immunoassay is enzyme-linked immunosorbent assay (ELISA).

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts HIC-HPLC separation and fractionation of a mixture of 2 mouse antibodies from Absolute Antibody (referred to as P13 and P14). The 4 rectangle area highlights 4 fractions that were reduced, digested and analyzed by LC-MS.

FIG. 2 shows a principal component analysis (PCA) of the different fractions from FIG. 1. Fractions were digested and analyzed by LC-MS. Antibody-1 is P13, Antibody-2 is P14.

FIG. 3 depicts a representative migration pattern of individual rabbit mAbs (referred to as P17, P18, P19 and P20) and the mixture (Mix) of the four mAbs on the native 7.5% PAGE. 10 μg of each mAb or 20 μg of the mixture was loaded per lane.

FIG. 4 depicts a PCA pairing analysis of rabbit mAbs mixture from the gel bands cut out of the gel on FIG. 3 reduced/alkylated, digested with a protease. The peptides were identified and quantified by LC-MS. The upper panel of FIG. 4 has been generated using 8 chains (4 light chains and 4 heavy chains of P17, P18, P19 and P20), while the bottom panel has been generated from the same dataset but using only 4 chains (2 light chains and 2 heavy chains P17 and P20).

FIG. 5 depicts a native 7.5% PAGE migration pattern of individual mouse mAbs (referred to as P12, P13, P14, P15 and P16) and the mixture of the five mAbs untreated (Mix) or treated with 0.5 μl or 1 μl citraconic anhydride (Mix+0.5CA or Mix+1CA), or sulfo-NHS-acetate (Mix+SNA). 10 μg of each mAb, 20 μg of the untreated or 30 μg of the treated mixture was loaded per lane. The total of 24 gel bands were cut out of the last four lanes, digested with chymotrypsin, and subjected to the pairing analysis.

FIG. 6 depicts a PCA pairing analysis of mouse mAbs mixture from the gel bands cut from the gel depicted in FIG. 5. The upper PCA plot was generated using 10 chains (5 light chains and 5 heavy chains of P12, P13, P14, P15 and P16), the bottom part of the figure has been generated using 4 chains only (2 light chains and 2 heavy chains of p12 and p16) as well on fewer fractions in order to resolve separation between those 2 antibodies.

FIG. 7 depicts a sequence alignment of the light and heavy chains from two recombinant antibodies named R1 and R3 identified from a polyclonal mix that was separated in a 2D gel (FIG. 8). The sequences in bold are peptides that have been selected as “proteotypic” and unique to each form and were used for co-expression analysis. R1 light chain=SEQ ID NO:27; R3 light chain=SEQ ID NO:28; R1 heavy chain=SEQ ID NO:29; R3 heavy chain=SEQ ID NO:30.

FIG. 8 depicts a 2D-gel of a rabbit polyclonal antibody under non-reducing conditions (7 cm IPG pH3-10NL; 4-20% SDS-PAGE) dominating by the R1 and R3 forms described from FIG. 7.

FIG. 9 shows an example of intensity correlation between different peptides across spots 1 to 12 (FIG. 8). Intensities are ArcSinH transformed prior to evaluate intensity correlation. Upper left is the correlation between peptide A and B (2 peptides from antibody R3 heavy chain (R3H) with a correlation coefficient of 0.9558), center is between peptide A and D (R3 heavy and R3 light with s correlation coefficient of 0.7157), and right is between peptide A and H (R3 heavy and R1 light with a correlation coefficient of −0.063). Bottom panel is the co-expression matrix between the 9 different selected proteotypic peptides from R1 and R3. Co-expression is observed only within heavy and light chains of both R1 and R3.

FIG. 10 is a graph showing the separation of the mix of 4 rabbit monoclonal by Imaged Capillary Isoelectric Focusing (iCIEF) and the 6 collected fractions. Those fractions were then digested and analyzed on Orbitrap Exploris 240.

FIGS. 11A-11D are graphs showing the peptide elution profiles across the different fractions obtained following iCIEF. FIG. 11A: Peptides from Heavy and Light chain from antibody R1. FIG. 11B: Peptides from Heavy and Light chain from antibody P18. FIG. 11B: Peptides from Heavy and Light chain from antibody P19. FIG. 11B: Peptides from Heavy and Light chain from antibody P17.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The present disclosure provides a method for pairing the Heavy (H) and Light (L) chains of an antibody present in an antibody mixture that does not necessitate the complete exact L and H chain sequences and allows pairing of specific short H chain fragments with specific short L chain fragments in particular their respective CDR3. The method relies on either separating the antibody mixture under non-reducing condition using chromatography (HIC or reverse phase or any separation technique) or gel (native gel or denaturing gel, 1D- or 2D-gel), or using cross-linking reagents and protease digestion to identify close proximity signature peptides from both H and L chains. Different data analysis strategies may be used to confirm the right pairings or to reduce the number of possible pairings.

The present disclosure provides a method for determining heavy and light chain pairing of an antibody or an antibody fragment in an antibody and/or antibody fragment mixture, the method comprising (a) submitting the mixture to a separation step under non-reducing condition to obtain isolated antibodies or antibody fragments; (b) digesting the isolated antibodies or antibody fragments (e.g., with a chemical cleavage reagent such as cyanogen bromide (CNBr) and/or one or more proteases) to obtain antibody or antibody fragment peptides; (c) analyzing the antibody or antibody fragment peptides; and (d) determining heavy chain and light chain pairing of the antibody or antibody fragment based on the analysis.

The present disclosure also provides a method for determining heavy and light chain pairing of an antibody or an antibody fragment in an antibody and/or antibody fragment mixture, the method comprising (a) contacting the mixture with a cross-linking agent to obtain cross-linked antibodies or antibody fragments; (b) digesting the cross-linked antibodies or antibody fragments (e.g., with a chemical cleavage reagent such as CNBr and/or one or more proteases) to obtain antibody or antibody fragment peptides; (c) analyzing the antibody or antibody fragment peptides; and (d) determining heavy chain and light chain pairing of the antibody or antibody fragment based on the analysis.

The expression “determining heavy and light chain pairing of an antibody or an antibody fragment in an antibody and/or antibody fragment mixture” means (a) identifying the exact heavy and light chain pairing of an antibody or antibody fragment, or (b) reducing the number of possible heavy and light chain pairing. For example, if a mixture comprises 10 antibodies (i.e., 10 light and 10 heavy chains sequenced), using to the method of Guthals et al. 2016, it would be necessary to recombinantly produce in cells and validate antigen binding for all 100 possible pairwise combinations to determine the proper pairing. However, the method of the present disclosure may permit to directly identify the proper pairing without the need to recombinantly produce in cells and validate antigen binding for all 100 possible pairwise combinations, or may limit the number of possible pairwise combinations to be produced by identifying light and heavy chains that cannot pair together because they were clearly found in distinct bands or fractions following separation under non-reducing conditions. Only light and heavy chains that were found in (c) under non-reducing conditions (i.e., in overlapping bands or fractions) would require further analysis (e.g., recombinant production in cells and assessment of antigen binding) to identify or confirm proper heavy and light chain pairing.

The present disclosure provides methods that allow for direct determination of heavy chain and light chain pairing of an antibody or antibody fragment in a mixture, for example, in a sample containing intact polyclonal antibodies (pAbs), a sample containing intact monoclonal antibodies (mAbs), or a sample containing antibody fragments (which can be generated from pAbs or mAbs (e.g., Fab fragments and/or F(ab′)₂ fragments)).

The separation of the antibodies or antibody fragments under non-reducing conditions may be performed using any known methods of separation of proteins under non-reducing conditions (conditions that maintain the disulfide bridges between the light and heavy chains, e.g., in the absence of reducing agents such as beta-mercaptoethanol (β-ME), dithiothreitol (DTT) or tris (2-carboxyethyl) phosphine (TCEP)), including chromatography-based methods such as hydrophobic interaction chromatography (HIC), ion exchange chromatography (anion or cation exchange chromatography), reverse phase (RP) or size exclusion chromatography, capillary electrophoresis (e.g., imaged capillary isoelectric focusing, CEF), as well as gel-based methods such as native polyacrylamide gel electrophoresis (PAGE), and agarose gel electrophoresis (denaturing 2D agarose gel). This step permits to divide the antibodies or antibody fragments present in the mixture into several fractions, with each fraction (or gel band) comprising either a single antibody or antibody fragment, or in the case of mixtures comprising antibodies or antibody fragments having similar migration patterns, one or more of the fractions or gel bands may comprise more than one antibody or antibody fragment (e.g., 2, 3 or 4 antibodies or antibody fragments).

In an embodiment, the method of the present disclosure further comprises contacting the antibody and/or antibody fragment mixture with an agent that modifies the charge of a given amino acid molecule in a sequence specific manner (thus changing the migration pattern of the antibodies or antibody fragments. Such agents are well known in the art and include agents that add positive or negative charges on certain neutral residues, agents that react with negatively charged residues (e.g., glutamic or aspartic acids) to remove the negative charge or to replace it by a positive charge, as well as agents that react with positively charged residues (e.g., lysine or arginine) to remove the positive charge or to replace it with a negative charge. Examples of such agents include citraconic anhydride (CA), which reacts with primary amines (e.g., in the side chain of lysine) to form an amide bond with a terminal carboxyl group, as well as sulfo-NHS-acetate (SNA), which also reacts with primary amines and forms a stable, covalent amide bond.

In an alternative embodiment of the method, the antibody and/or antibody fragment mixture is contacted with a protein cross-linking agent to obtain cross-linked antibodies or antibody fragments. The objective of this step is to generate covalent bonds between the heavy and light chains. The protein cross-linking agent may be a bifunctional reagent or heterofunctional reagent. The reactive function of the protein cross-linking agent can be an NHS ester compound (that reacts with primary amine), a maleimide compound (that reacts with sulfhydryl containing molecule), an hydrazide compound (that reacts with aldehyde containing molecule), or a carbodiimide based compound such as EDC (that reacts with carboxylate containing molecule). The two functional groups may be separated by a spacer, such as an alkyl or polyethylene glycol (PEG) chain. Agents to induce protein cross-linking are well known in the art and include, for example, glutaraldehyde, DSG, disuccinimidyl suberate (DSS), Bis(sulfosuccinimidyl) suberate (BS3), Bis(succinimidyl) penta(ethylene glycol (BS(PEG)5), TSAT, DSP, DTSSP, DST, BSOCOES, EGS, Sulfo-EGS, DMA, DMP, DMS, DTBP, DFDNB, SIA, SMAP, SIAB, Sulfo-SIAB, AMAS, BMPS, GMBS, Sulfo-GMBS, MBS, Sulfo-MBS, SMCC, Sulfo-SMCC, SMBP, Sulfo-SMBP, SMPH, LC-SMCC, Sulfo-KMUS, SPDP, LC-SPDP, Sulfo-LC-SPDP, or SMPT.

In an embodiment, in cases where at least the partial amino acid sequence of the antibodies or antibody fragments is not known, the method of the present disclosure further comprises a step of determining the amino acid sequence of at least a portion of the light and heavy chains of the antibodies or antibody fragments present in the mixture (e.g., at least the heavy chain CDR3 and light chain CDR3). The sequences of the light and heavy chains of the antibodies or antibody fragments may be determined using methods well known in the art, such as de novo MS/MS sequencing (Guthals et al. 2016), high throughout Ig sequencing (Georgiou et al., 2014), proteogenomics-based sequencing (Hashimoto et al., 2020; Cheung et al., 2012, Wine et al., 2013), and single-cell sequencing (Meijer et al., 2006). Sequences of the heavy chain CDR3 may be determined using the method described in PCT application No. PCT/CA2021/050791. The method may also include collecting additional information such as the germline/allele, if sequencing successfully the entire antibody is not possible

After the separation of the antibodies or antibody fragments under non-reducing conditions, one or more of the fractions or gel bands (or the cross-linked antibodies or antibody fragments) are digested (under reduced and/or non-reduced condition) with a suitable agent to generate digested peptides proper for LC-MS analysis. Agents to cleave proteins include chemical agents such cyanogen bromide (CNBr) that cleaves at methionine (Met) residues; BNPS-skatole that cleaves at tryptophan (Trp) residues; formic acid that cleaves at aspartic acid-proline (Asp-Pro) peptide bonds; hydroxylamine that cleaves at asparagine-glycine (Asn-Gly) peptide bonds, and 2-nitro-5-thiocyanobenzoic acid (NTCB) that cleaves at cysteine (Cys) residues, as well as proteases. Any suitable protease or combination of proteases may be used to digest the isolated fractions or gel bands, such as trypsin, chymotrypsin, AspN, GluC, ArgC, LysC, LysN, pepsin or any combination thereof. In the case of cross-linked antibodies or antibody fragments, the digestion will generate peptides of both the light and heavy chains. The digestion may be performed with a combination of chemical agent(s) and protease(s).

Prior to and/or after the digesting step, the sample may be subjected to various treatments including alkylation and deglycosylation.

The antibody or antibody fragment peptides are then submitted to a suitable analysis to identify the composition of the heavy and light chain in each fraction (or gel band), and thus to determine the proper heavy and light chain pairing in the mixture. For example, the peptides could be resolved by reverse phase chromatography and in-line nanoelectrospray ionization/high-resolution tandem mass spectrometry (MS/MS). In an embodiment, the analysis comprises submitting the antibody or antibody fragment peptides to mass spectrometry (MS), such as liquid chromatography-mass spectrometry (LC-MS), preferably tandem MS. The MS/MS profile of the antibody or antibody fragment peptides may be compared to databases of antibody peptide sequences, or de novo sequencing of at least a portion of the antibodies or antibody fragments in the mixture may be performed.

The analysis may also comprise performing a quantitative analysis of the peak intensity of the antibody or antibody fragment peptides. The analysis comprises performing a clustering analysis or cross-correlation analysis to identify peptides that are found at similar level/frequency in the same fraction or gel band. Suppose there are n fractions or gel bands. Each peptide P⁽ⁱ⁾ is then associated with a quantification vector X⁽ⁱ⁾=(x₁⁽ⁱ⁾, x₂⁽ⁱ⁾, . . . , xₙ⁽ⁱ⁾, where each x) is the normalized quantity of peptide P⁽ⁱ⁾ in fraction (or gel band) j. Since the initial fractionation is at the protein level, peptides from the same antibody or antibody fragment should have very similar quantification vectors. In contrast, the quantification vectors of peptides from different antibodies or antibody fragments are usually different because their corresponding proteins are likely separated into different fractions (or gel bands) or appear in different fractions (or gel bands) at different concentration. As such, a clustering analysis using the quantification vector may cluster the peptides according to their corresponding proteins. The peptides belonging to the same cluster are then used together to assemble one or more protein sequences. Such clustering can also be used to pair the corresponding H and L chains of the same antibody or antibody fragment. Essentially, the chains whose peptides mostly belong to the same cluster are paired together.

The clustering analysis can be carried out with any standard clustering algorithms widely known to the data analysis community, such as K-means clustering and Hierarchical clustering that are available through software packages such as R (r-project.org) and scikit learn (scikit-learn.org). A standard principal component analysis (PCA) can also be used to reduce the dimensionality of the quantification vectors before the clustering analysis. PCA is available in many standard data analysis software packages such as R and scikit learn. When the number of antibodies or antibody fragments is small (e.g., less than 5), a PCA can also be used to project the vectors to a two- or three-dimensions space so the data can be visualized to determine the clustering or to pair the H and L chains. In an embodiment, the clustering analysis is a principal component analysis (PCA).

Analysis of cross-linked peptides may be performed using suitable tools such as pLink (Fan et al. 2015), ECL (Yu et al., 2016), xQuest (Rinner et al., 2008; Walzthoeni et al., 2012), ProteinProspector (Chu et al., 2010; Trnka et al., 2014), Kojak (Hoopmann et al., 2015), OpenPepXL (Netz et al., 2020) or MS-Annika (Pirklbauer et al., 2021).

In an embodiment, the method further comprises assessing the binding of the antibody or antibody fragment to its target antigen. This step may be used to confirm that the putative heavy and light chain pair identified by the method form a functional antibody or antibody fragment, or in the case where more than one putative/candidate heavy and light chain pairs are identified (e.g., when two or more antibodies or antibody fragments in the mixture have similar migration patterns), this step permits to confirm which of the putative/candidate heavy and light chain pairs forms a functional antibody or antibody fragment able to bind to the target antigen. This may be done, for example, by recombinantly expressing an antibody or antibody fragment comprising the putative heavy and light chain pair identified, and assessing the binding of the antibody or antibody fragment to its target antigen. This may be achieved by introducing nucleic acids encoding the heavy and light chains into a suitable expression system, such as CHO or HEK293 cells, culturing the cells into conditions suitable for the production of the antibody or antibody fragment, and assessing the binding of the antibody or antibody fragment to the antigen, for example by immunoassay (e.g., ELISA). In an embodiment, the binding of the antibody or antibody fragment to its target antigen may be assessed by expressing the antibody or antibody fragment at the surface of a phage and assessing the binding of the phages to the antigen (phage panning).

### EXAMPLES

The present disclosure is illustrated in further details by the following non-limiting examples.

### Example 1: Chromatographic Separation of 2 Antibodies Under Non Reducing Conditions and H/L Pairing Based on Peptide Identification and Principal Component Analysis (PCA)

The aim of this study was to properly pair two mouse IgG2a from an artificial mixture using hydrophobic interaction chromatography (HIC). The two antibodies used were two mouse IgG2a recognizing two different antigens (Absolute Antibody, referred to as P13 and P14).

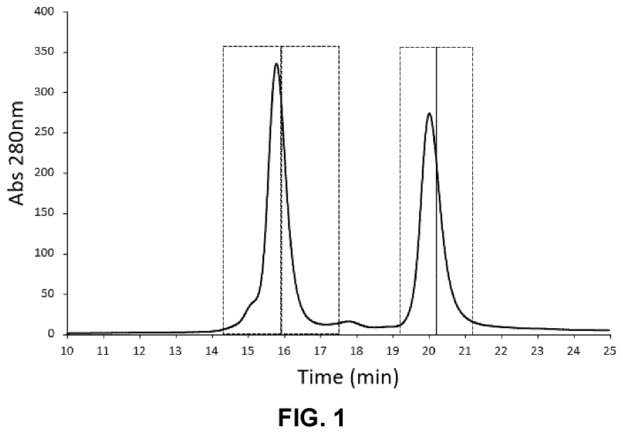

The HIC column used is a Propac 2.1 mm×100 mm Thermo Fisher #063653. 50 μg of each intact antibody were mixed and separated on a 35-minute gradient. In FIG. 1, two main peaks were detected at 15.7 min and 20 min. Each of those peaks was initially fractionated into 4 fractions each (total 8 fractions named 1 to 8). Fractions were then pooled by pair (fraction 1 and 2 pooled, then 3-4, 5-6 and 7-8). A buffer exchange was performed using Amicon® Pro Purification System with Amicon® Ultra-0.5 Device to replace the ammonium sulfate present in the HIC elution with PBS. The resulting 4 pooled fractions were then reduced and alkylated and digested using the 2 proteases chymotrypsin and pepsin. The digested fractions were analyzed by LC-MS using an EVOSEP coupled to a Q Exactive in data dependent mode (44-minute gradient per run).

Two different analyses were performed to confirm the proper H and L chain pairing from that mixture.

Analysis 1: To identify the composition of the H and L chain in each fraction, the mass spectrometry peptides were compared to an internal database of all in-house antibodies.

TABLE 1
Peptide count per fractions
Peak 1 (15.7 min): pooled fraction 1-2 and 3-4

| | PSM | #peptides |
|---|---|---|
| P13H | 1014 | 353 |
| P13L | 520 | 187 |
| P14H | 20 | 14 |
| P14L | 11 | 6 |

Peak 2 (20.0 min): pooled fraction 5-6 and 7-8

| | PSM | #peptides |
|---|---|---|
| P13H | 82 | 42 |
| P13L | 77 | 39 |
| P14H | 812 | 271 |
| P14L | 487 | 180 |

PSM: Peptide Spectrum Matches

Conclusion based on PSM and peptides specificity: analysis of the number of hits per peak show that Peak 1 at 15.7 min contain mostly the H and L chain of P13 while Peak 2 show a higher content for the H and L chain for P14 (as shown by the number in italics). Thus, HIC can separate antibodies and its peptide spectrum matches can be used to pair correctly the heavy and light chain of P14 and similar for P13.

Analysis 2: To identify the proper pairing of the heavy and light chain in each fraction, principal component analysis (PCA) of the peptides trend across the 4 fractions (2 fractions per peak) was performed.

The same experimental dataset described above was analyzed using a principal component analysis. Suppose there are n fractions. Each peptide P(O is then associated with a quantification vector X⁽ⁱ⁾=(x₁⁽ⁱ⁾, x₂⁽ⁱ⁾, . . . , xₙ⁽ⁱ⁾), where each x) is the normalized quantity of peptide P⁽ⁱ⁾ in fraction j. Since the fractionation is at the protein level, peptides from the same mAb protein should have very similar quantification vectors. In contrast, the quantification vectors of peptides from different mAb proteins are usually different because their corresponding proteins are likely separated into different fractions or appear in different fractions at different concentration. As such, a clustering analysis using the quantification vector may cluster the peptides according to their corresponding proteins. The peptides belonging to the same cluster are then used together to assemble one or more protein sequences. Such clustering can also be used to pair the corresponding H and L chains of the same mAb. Essentially, the chains whose peptides mostly belong to the same cluster are paired together.

The clustering analysis can be carried out with any standard clustering algorithms widely known to the data analysis community, such as K-means clustering and Hierarchical clustering that are available through software packages such as R (r-project.org) and scikit learn (scikit-learn.org). A standard PCA can also be used to reduce the dimensionality of the quantification vectors before the clustering analysis. PCA is available in many standard data analysis software packages such as R and scikit learn. When the number of mAbs is small (e.g., less than 5), a PCA can also be used to project the vectors to a two- or three-dimensions space so the data can be visualized to determine the clustering or to pair the H and L chains.

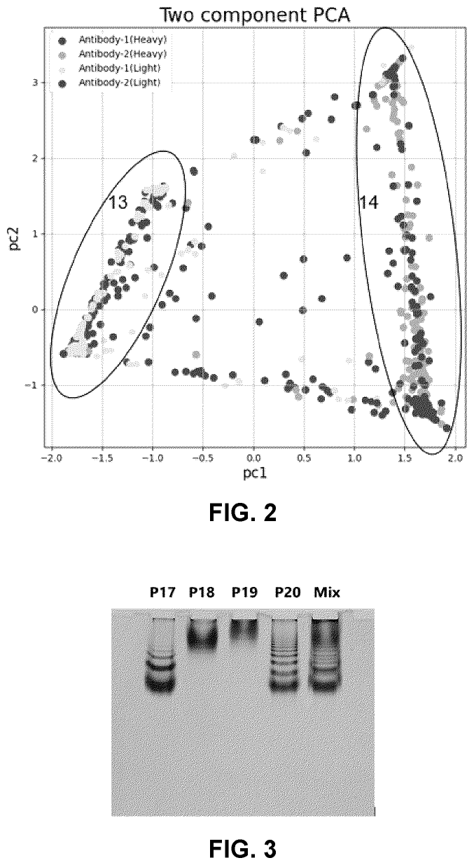

For example, FIG. 2 shows the PCA analysis to pair the H and L chains of a two-antibody mixture. In this experiment, four sequences (H1, H2, L1, and L2) are first determined through other means. Then the quantification vectors of the peptides unique to each chain are projected into a two-dimensional space using a PCA analysis and plotted in the figure. The four colors correspond to the four chains. It can be seen that the correct pairing should be (H1, L1) and (H2, L2) as their peptides are clustered together in the PCA plot.

In the PCA plot, there are no clustering of the two Light or the two Heavy chains but instead two main clusters of one pair each of H with L chains, antibody 1/P13 H and L chains on the left and antibody 2/P14 H and L chains on the right. In PC2, a homogeneous distribution of H and L for both antibodies is seen.

### Example 2: Native Gel Electrophoresis to Separate 4 Antibodies Under Non Reducing Conditions and H/L Pairing Based on PCA Analysis

Proteins loaded on polyacrylamide gel electrophoresis (PAGE) separate according to their charge, shape and molecular weight. Antibody proteins have similar molecular weights and shapes but could differ from each other by their charge. Several factors contribute to the overall charge of the Ig molecule, including the abundance of amino acids with basic and acidic side chains, and the amount and composition of glycans. Pure mAbs subjected to native PAGE will thus exhibit varying degrees of migration into the gel. In addition, mAbs band pattern will often contain multiple bands, presumably reflecting the degree of glycosylation.

The pairing strategy in this example takes advantage of the distinctive migration pattern of rabbit mAbs on native PAGE. The strategy comprises separating the rabbit mAb mixture under native (non-denaturing, non-reducing) conditions on PAGE, digesting the proteins in the cut gel bands with the protease, followed by the LC-MS/MS analysis of the peptides and the computer algorithm matching the unique peptides originating from the H and L chains found in the same gel band.

Four rabbit IgG recombinant antibodies (anti-beta 3 integrin (P17), anti-ERBB2 (P18), anti-IL-18 (P19), and anti-alpha-Vp5 integrin (P20), each at 1 mg/mL) from Absolute Antibody with known amino acid sequences were mixed in equal proportions. Twenty μg of the mixture was mixed with the 2× native loading dye and loaded on the 7.5% PAGE. In parallel, pure antibodies (10 μg each) were loaded on the same gel for comparison. The separation was performed at 130V for 1 H 15 min. The gel was then stained with Coomassie Blue dye and de-stained with Methanol/Acetic acid to visualize the antibodies band pattern (FIG. 3). The total of 9 bands were then cut from the gel lane containing the mixture of mAbs (Mix) and subjected to chymotrypsin in-gel digestion protocol. Briefly, the gel bands were de-stained with 50% methanol and dehydrated with acetonitrile (ACN). The proteins in the bands were then reduced with 25 mM DTT for 30 min at 56° C., followed by cysteine alkylation with 55 mM IAA for 30 min in the dark. The bands were then washed, dehydrated with acetonitrile, and air dried. The chymotrypsin digestion of proteins in each gel band was performed with 50 μl of 12 ng/μl enzyme in the presence of 0.01% ProteaseMax© Surfactant for 2 hours at 37° C. The peptides were extracted from the gel bands with 60% ACN/0.1% FA with sonication for 30 min, dried in Speed-Vac and reconstituted in 0.1% FA for MS analysis.

The MS analysis was performed on Orbitrap Fusion™ Tribrid™ Mass Spectrometer coupled to Evosep One LC system.

The data were analyzed using PCA as described above in Example 1, analysis 2. The consistent finding of the unique peptides from H and L chains in the same fraction identifies a cognate H-L chain pair. Unique peptides to each antibody have a distinctive pattern across the different fractions. The best correlation pattern between a given H and L chain allows for proper pairing.

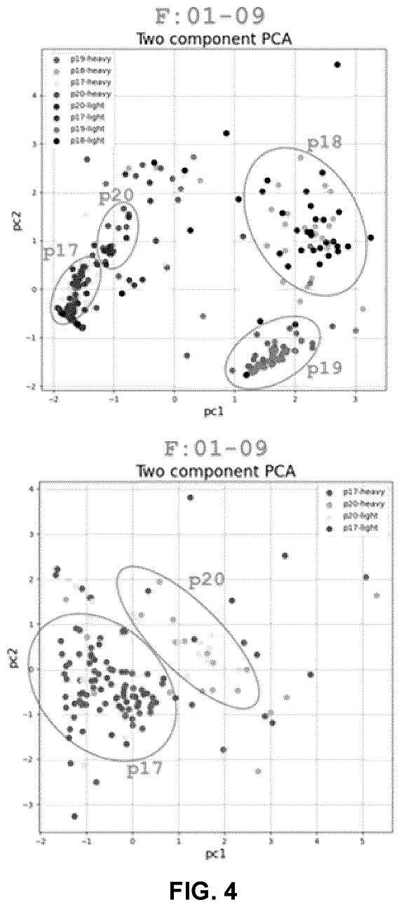

FIG. 3 shows that each of the four rabbit mAbs had a relatively distinct migration pattern in native PAGE, allowing sufficient separation of the individual antibodies (thus the different H-L pairs) from the mixture. The PCA could thus accurately match the proper H and L chains for each individual mAb in the artificial mixture (FIG. 4).

### Example 3: Native Gel Electrophoresis to Separate 5 Labelled Antibodies Under Non Reducing Conditions and H/L Pairing Based on PCA Analysis

Complex mixture of mAbs with similar migration patterns on native PAGE may not be efficiently separated into individual mAbs and thus may not be accurately paired by PCA. In addition, some mAbs do not migrate well in the gel resulting in the inability to generate data for pairing. In this example, a pairing strategy that takes advantage of mAb labeling that modifies the charge of the protein molecule in a sequence specific manner. The main principle consists of changing the migration pattern of the mAb on the native PAGE. This labelling strategy was used to solve these issues and pair up to 5 different mouse recombinant Abs. Two compounds, citraconic anhydride (CA) and sulfo-NHS-acetate (SNA), reacting with primary amines were selected as modifying agents.

Citraconic anhydride (or 2-methyl maleic anhydride) reacts with primary amines to form terminal carboxylate under neutral pH. Under acidic conditions (pH 3-4) the reaction is reversible. Introduction of a carboxylate group contributes a negative charge and results in a net charge change of −2 per modified amine group. It was found that the reaction efficiency depends on the pH and the amount of citraconic anhydride added. If the product of CA reaction is then separated on the native PAGE, the observed migration pattern would be different depending on the specific conditions of the reaction. Two labeling conditions produced recognizable and distinct migration patterns for mAbs mixture on the native PAGE: reaction with 0.5 μl CA and reaction with 1 μl CA. The reversible nature of the reaction is advantageous, since peptide separation prior to MS analysis is done under acidic conditions that favor the release of the free amine group. Thus, no specific amino acid modification needs to be considered at the data analysis level.

Sulfo-NHS-acetate is commonly used to block or protect primary amines. The reaction with amine groups of proteins at neutral pH results in the formation of non-reversible acetamide modifications. Acetylation of lysines and free primary amines, reduce the overall protein charge under neutral and acid condition. Indeed, the reaction with sulfo-NHS acetate also changes the migration pattern of the modified protein on the native PAGE. The acetylation of lysine residues adds 42.01056 amu, which is taken into account at the data analysis.

Both CA and sulfo-NHS-acetate modify N-terminus of the protein as well as lysine residues. Since trypsin, Lys-C and in some instances Lys-N, does not cut after modified lysine residues, none of those enzymes were chosen for the enzymatic digestion of the modified proteins. Instead, pepsin or chymotrypsin were used.

Five mouse IgG2A mAbs recognizing different antigens (referred to as P12, P13, P14, P15 and P16, each at 1 mg/mL, all from Absolute Antibodies) with known amino acid sequences were mixed in the equal proportions by mixing 75 μl aliquot of each mAb. The resulting mixture was split into 4 treatments:
1. 75 μl aliquot was left untreated
2. 100 μl aliquot was mixed with 150 μl of 0.1M sodium carbonate buffer (pH 9) and 0.5 μl of citraconic anhydride (mix+0.5 CA).
3. 100 μl aliquot was mixed with 150 μl of 0.1M sodium carbonate buffer (pH 9) and 1 μl of citraconic anhydride (mix+1CA).
4. 100 μl aliquot was mixed with 150 μl of 0.1M PBS buffer (pH 7.2) and 1-2 crystals of Sulfo-NHS-Acetate (mix+SNA).

All treated samples (conditions 2-4) were then incubated at 25° C. for 2 hours with mixing, followed by overnight incubation at +4° C. 125 μl aliquot (50 μg) of each treated sample was desalted on Zeba desalting column with 7.5 kDa MWCO and dried under low pressure centrifugation (SpeedVac™). The dry pellet was reconstituted in 25 μl of 50 mM ammonium bicarbonate buffer and mixed with 25 μl of 2× native loading dye. 30 μl (30 μg) of the mixture was then loaded on the 7.5% polyacrylamide gel. In parallel, 20 μg of untreated mixture as well as 10 μg of each individual mAb were loaded on the same gel. The native PAGE separation was performed as described in Example 2. Upon the separation on the gel, the bands were cut out of the untreated and treated mAbs mixtures as follows: bands 1-8 were cut from untreated mixture, 9-13 were cut from sample treated with 0.5 μl of CA, 14-19—from sample treated with 1 μl of CA, 20-24—from sample treated with sulfo-NHS-acetate. The cut gel bands were then digested with chymotrypsin and analyzed with LC-MS/MS as described in the Example 2.

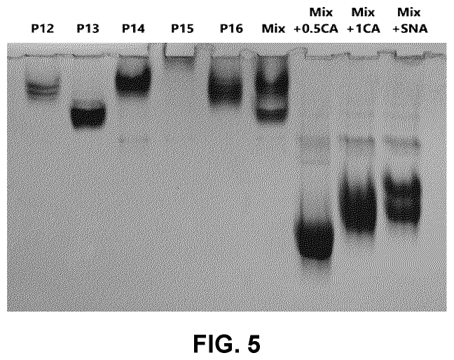

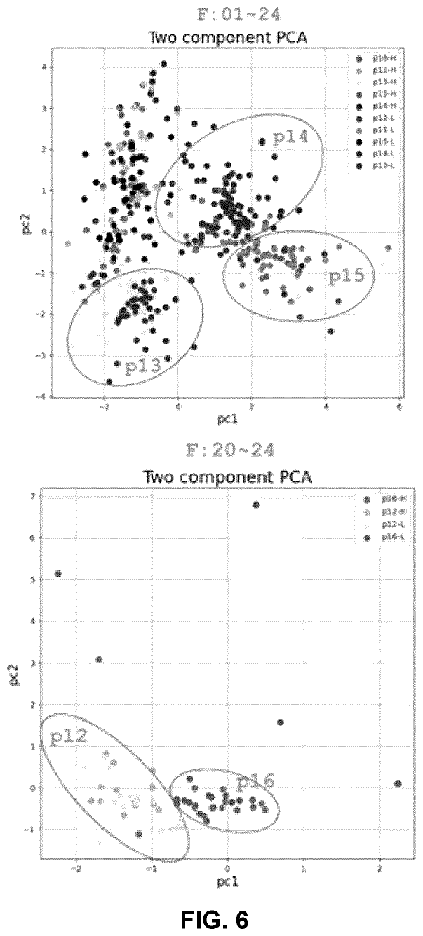

FIG. 5 shows that the migration pattern of untreated mouse mAbs mixture is a combination of the migration patterns of the individual mouse mAbs with a poor degree of separation. However, upon treatment with citraconic anhydride (CA) or sulfo-NHS-acetate (SNA), the migration pattern of the mixture changes as labelled proteins migrate significantly more in the gel. It is expected that the migration of individual mAbs in the mixture relative to one another is also affected by the treatments, since each mAb has varying number of lysine residues available for modification. FIG. 6 demonstrates that the pairing of P13, P14, P15 could be achieved when all untreated and treated mixture fractions are considered, however P12 and P16 remain unresolved under these conditions. The accurate pairing of P12 and P16 was achieved with separate evaluation of mixture labeled with SNA or 0.5 μl CA.

### Example 4: Denaturing 2D Gel Under Non-Reducing Condition to Separate the Different Components of Natural Rabbit Polyclonal Antibodies and H/L Pairing Based on Co-Expression Analysis of Few Proteotypic Peptides

For this example, a natural rabbit pAbs directed against the JC region adjacent to the CDR3 human H chain (referred to as internally as a rabbit polyclonal antibody with the naming “PD025”) was used.

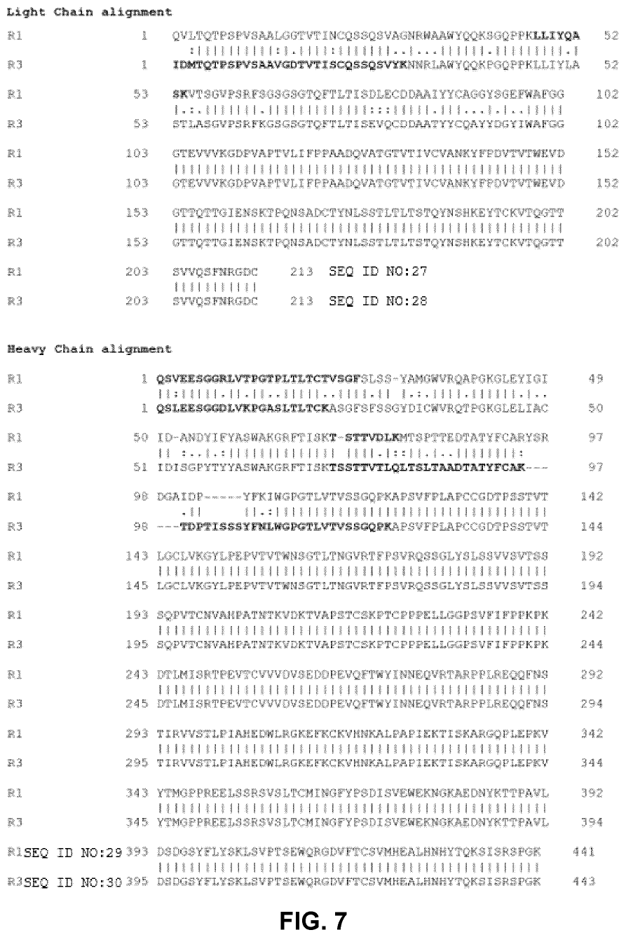

As a proof of concept, the natural rabbit pAb PD025 was separated under non-reducing condition using a 2D gel, followed by in-gel trypsin digestion. In this natural pAb, 2 main antibodies, named R1 and R3, were identified. Their sequence alignments are shown in FIG. 7.

As stated earlier, it is assumed in that example that R1 and R3 H and L chains were assembled and only the right pairing was missing. As shown in FIG. 7, some differences were observed in the sequence of the F_{ab} region for both H and L chains. Nine peptides unique to each form of R1 and R3 and L chains have been identified and selected (Table 2). In FIG. 7, the different unique peptides that are used to distinguish each R1 and R3 H and L chains are shown in bold. A total of 9 peptides have been selected and are shown in Table 2, 5 peptides for R3 antibody and 4 peptides for R1 antibody. They have been selected based on 2 criteria: 1) they are proteotypic and 2) they generate intense MS peak intensity. One of the peptides from the heavy chain of R1 named “G” is also a non-tryptic peptide.

TABLE 2

| Naming | # | m/z | z | Sequence | Comments |
|---|---|---|---|---|---|
| R3H | A | 957.8263 | 3+ | TDP...GQPK (Residues 98-125 of | |
| | | | | SEQ ID NO: 21) | |
| R3H | B | 884.7923 | 3+ | TSS...CAK (Residues 73-97 of SEQ | |
| | | | | ID NO: 21) | |
| R3H | C | 758.3992 | 3+ | QSL...TCK (Residues 1-22 of SEQ ID | |
| | | | | NO: 21) | |
| R3L | D | 1053.196 | 3+ | IDM...VYK (Residues 1-30 of SEQ ID | |
| | | | | NO: 19) | |
| R3L | E | 831.0024 | 2+ | LLI...PSR (Residues 47-62 of SEQ ID | |
| | | | | NO: 19) | |
| R1H | F | 466.2187 | 2+ | QSV...GGR (Residues 1-9 of SEQ ID | |
| | | | | NO: 20) | |
| R1H | G | 882.4837 | 2+ | LVTP...SGF (Residues 10-26 of SEQ | Not tryptic. |
| | | | | ID NO: 20) | |
| R1L | H | 486.2919 | 2+ | LLIYQASK (Residues 47-54 of SEQ | |
| | | | | ID NO: 18) | |
| R1H | — | 432.7461 | 2+ | TSTTVDLK (Residues 72-79 of SEQ | |
| | | | | ID NO: 20) | |

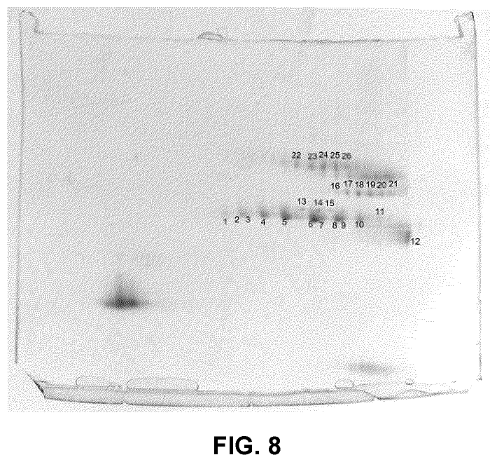

Assuming the different H and L chains are either fully assembled or specific proteotypic peptide identified for each chain (i.e., the respective L and H chains sequences are complete or partly complete but not paired), the proposed strategy includes identifying unique peptides to each H and L chains. The mixture was separated under non-reducing condition using 2D gel (FIG. 8), all spots were digested and analyzed by LC-MS. The intensity of each proteotypic peptide was measured across the different fractions, and the correlated intensities profiled between H and L chains.

The antibody PD025 was separated using a 2D gel under non-reducing condition as following. For sample cleanup, 150 μg was precipitated using TCA (25%) at 4° C. overnight to remove glycerol and salt. The sample was centrifuged at 27,237×g at 4° C. for 30 min. The pellet was washed with cold acetone twice, centrifuged at 27237×g at 4° C. for 10 min between each wash step. Then, the pellet was resuspended in 130 μl DeStreak™ Rehydration Solution (Cytiva) with 1.3 μl BioLyte® 3/10 Ampholyte (Bio-Rad). The resuspended sample was used to passively rehydrate a 7 cm IPG strip (pH3-10NL, Bio-Rad) overnight at room temperature Next day, isoelectric focusing was conducted in a PROTEAN® IEF Cell (Bio-Rad) at 250 V 20 min, 4,000 V 2 h, and 4,000 V to 10,000 V-h with the current set at 50 μA per gel. When the IEF separation was completed, the IPG strip was removed from the focusing tray and transferred side up onto a blotting filter paper. For non-reducing sample, the IPG strip was equilibrated in SDS Equilibration buffer [6M urea; 0.375M Tris-HCl, pH 8.8; 2% SDS; 20% glycerol] for 20 min at room temperature. Next, the IPG strip was equilibrated in 1× Tris-glycine-SDS running buffer before it was sealed onto a 4-20% Mini-PROTEAN® TGX™ Precast gel (Bio-Rad) using agarose solution [0.5% low melting point agarose in 1× Tris-glycine-SDS and 0.003% bromophenol blue]. The gel was stained using Coomassie Brilliant Blue R-250 (see FIG. 8). All spots were digested with trypsin using standard procedure and analyzed using an LC-MS Evosep-Q Exactive™ platform, 44 minutes.

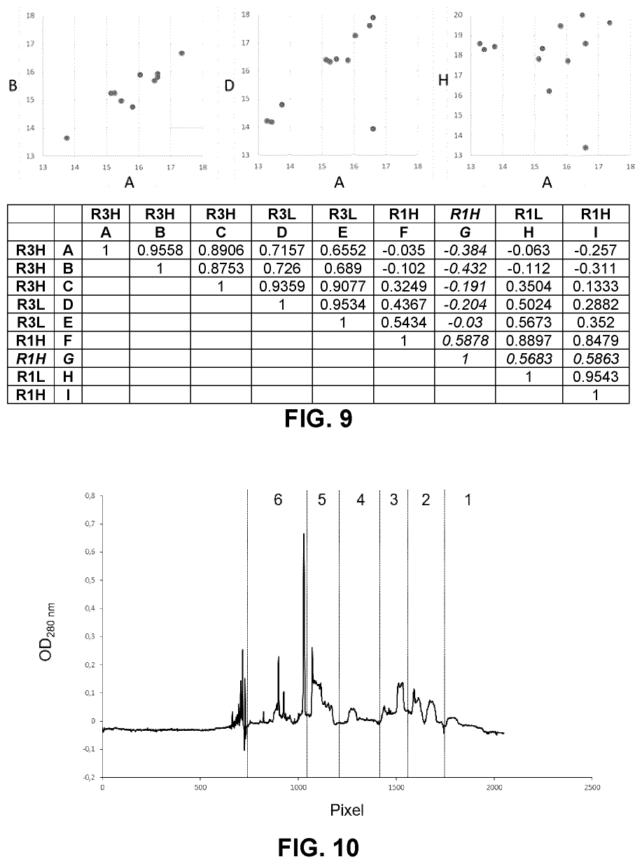

The peak intensities for the 9 selected peptides described in Table 2 were evaluated for fractions 1 to 12 (see FIG. 8). The intensity data for the selected peptides was transformed using an ArcSinH transformation function which allows the analysis of “0” or null intensity contrary to log scale. Scatter correlation was evaluated between each peak across the 12 fractions as shown in the top of FIG. 9. The scatter plot on the left is the correlation between a peptide from R3H (peptide A) with another peptide from R3H and show a correlation of 0.9558. A good correlation is also found between peptide A and peptide D (R3L) with a correlation of 0.7157. A poor correlation is found between peptide A R3H and a peptide from light chain from R1 (peptide H) at −0.063. A global correlation matrix is shown at the bottom of FIG. 9. The overall matrix shows good correlation between peptides from a given recombinant form. Such an approach can be used to pair heavy and light chains properly for both R1 and R3.

### Example 5: Identification of Heavy and Light Chain Pairing Using a Cross-Linker

Crosslink approach has been used in the field of IgG characterization for a while to map antibody/antigen contact. Maibom-Thomsen et al. (2019) have shown using crosslink that IgG has a compact structure with a hidden Fc domain. Most of the work done on using crosslink and IgG are either toward coupling covalently the antibody to protein A/G beads or to study the epitope sites.

100 μg of each of R1 and R3 were combined, followed by addition of 4 μL 50U of the protease IdeS derived from Streptococcus pyogenes (Promega), and dilution up to 100 μL with 0.01M PBS. The mixture was incubated at 37° C. for 1 hour. 60 μL of protein A slurry was washed with 500 μL 0.01M PBS for R1/R3 mix and incubated for 1 hour at room temperature and mix. The supernatant, containing F(ab′)₂ fragments, was retained. 60 μg of F(ab′)₂ was dried down using centrifuge under low pressure. 30 μg R1 and 30 μg R3 undigested (“intact” fraction) were combined, then dried down under low pressure. R1/R3 F(ab′)₂ were reconstituted in 20 μL 400 μM bis(sulfosuccinimidyl)suberate (BS3, Thermo Fisher) dissolved in 25 mM sodium phosphate (20× molar excess of BS3 to R1/R3 F(ab′)₂). Intact R1/R3 mix was reconstituted in 50 μL 280 μM BS3 dissolved in 25 mM sodium phosphate (35× molar excess of BS3 to R1/R3 mix). The crosslinking reaction was allowed to take place at room temperature on shaker for 1 hour. The reaction was quenched by adding 1 M tris to a final concentration of 60 mM. F(ab′)₂ and intact R1/R3 were diluted to 50 μL using 0.01M PBS followed by addition of 1.5 μL 1 M DTT to each and incubation at 95° C. for 15 minutes. 17.2 μL was removed for cysteine modification. 30 μL of 0.5M 2-Bromoethylamine hydrobromide (BEA) solution as well as 10 μL 1M tris pH 8 were added to volume set aside for cysteine modification (Cet). The reaction was incubated at room temperature for 4 hours, and 1M tris was added every hour to maintain reaction pH at 7. The remaining 32.8 μL was alkylated with 5 μL 0.5M iodoacetamide for 30 minutes at room temperature in dark. 170 μL acetone was added to iodoacetamide-treated samples, and 300 μL acetone was added to cysteine-modified samples for 1 hour at −20° C. The mixtures were centrifuged at 27,237×g at 4° C. for 10 minutes, and the acetone was decanted. Any remaining acetone was dried using centrifuge under low pressure. The pellet was reconstituted in 4 μL 4M urea in 37° C. shaker for 10 minutes. Cysteine-modified sample was then diluted up to 20 μL using HPLC grade water, and 30 μL 50 mM ammonium bicarbonate was added. 1 μg trypsin was and incubated overnight at 37° C. The iodoacetamide-treated sample was diluted up to 40 μL using HPLC grade water then split 20 μL into two tubes. 30 μL 50 mM ammonium bicarbonate and 1 μg of chymotrypsin were added to one tube and 25 μL 0.04 μg/μL pepsin (1 ug) plus 2 μL HCl were added to the second tube. Pepsin digest was performed at 37° C. for 15 minutes, followed by inactivation at 95° C. for 3 minutes, and the mixture was dried down with centrifuge under low pressure. Following overnight digestion, samples were dried down under low pressure centrifugation (Speedvac™). Digests were reconstituted in 40 μL 0.1% FA to achieve sample concentration of 0.5 μg/μL. The digest was loaded on Evotips following manufacturer's instruction.

5 μg of each digest (pepsin, chymotrypsin and cysteine modified followed by trypsin digestion) were loaded for both F(Ab′)₂ and intact samples in 6 tips, followed by centrifugation at 1000×g for 1 minute. The tips were washed by adding 25 μL 0.1% FA in water and spinning at 700×g for 1 minute. The tips were left in 200 μL 0.1% FA by spinning down briefly. The samples were loaded on Q Exactive mass spectrometer using 44-minute LC-MS method.

The identification of BS3 cross-linked and mono-linked peptides was performed using pLink v2.3.9 software (Fan et al. 2015). The search parameters were set as follow: Trypsin (Try) or Pepsin (Pep) was selected as the protease, cysteines were set as fixed modification either carbamidomethyl-C or CEthy. As variable modifications, deamidated-N, deamidated-Q, oxidation-M, pyro-Glu at any Q N-term, and/or carbamidomethyl at any N-term were selected. The range of peptide mass and peptide length was set as 400-9000 Da and 4-90 aa, respectively. The cross-linked and mono-linked peptides were filtered using a <=5% FDR cut-off at PSM level and a pLink score <0.01.

The proof of concept of this study was to show that by mixing 2 known antibodies (from same species) and crosslinking the antibodies, it is possible to detect a unique crosslink peptide allowing the pairing of a given heavy chain with its corresponding light chain. By using a mixture, the importance of possible artefact (i.e., wrong crosslink pairs) was also evaluated. In Table 3, at least 7 crosslink peptides were identified for proper H/L pairing, 5 for R1 and 2 for R3. All other crosslinks were intrachain which do not allow to resolve H/L pairing. No interchain crosslink were found between R1 and R3.

TABLE 3
Crosslink peptides

| | | | | | Protein |
|---|---|---|---|---|---|
| # | Peptide | Mass | Modifications | Proteins | type |
| 1 | DGAIDPYFKIWGPGTLVTVSSGQPK(9)- | 4300.2902 | None | R1-H to | Inter |
| | SGQPPKLLIYQASK(6) (SEQ ID NOs: 1 and 2) | | | R1-L | |
| 2 | DGAIDPYFKIWGPGTLVTVSSGQPK(9)- | 4489.4015 | None | R1-H to | Inter |
| | LLIYQASKVTSGVPSR(8) (SEQ ID NOs: 1 | | | R1-L | |
| | and 3) | | | | |
| 3 | DGAIDPYFKIWGPGTLVTVSSGQPK(9)- | 5083.7141 | None | R1-H to | Inter |
| | SGQPPKLLIYQASKVTSGVPSR(14) (SEQ ID | | | R1-L | |
| | NOs: 1 and 4) | | | | |
| 4 | QVLTQTPSPVSAALGGTVTINCQSSQSVAGNR(1)- | 5807.8297 | CEthy(22); | R1-H to | Inte |
| | TSTTVDLKMTSPTTEDTATYFC(8) (SEQ ID | | Cethy(57) | R1-L | |
| | NOs: 5 and 6) | | | | |
| 5 | LLIYQASKVTSGVPSRFSGSGSGTQ(7)- | 3422.7953 | None | R1-H to | Inter |
| | KIWGPGTL(1) (SEQ ID NOs: 7 and 8) | | | R1-L | |
| 6 | IDMTQTPSPVSAAVGDTVTISC(1)- | 3645.8422 | CEthy(22); | R3-L to | Inter |
| | QTPGKGLELIAC(5) (SEQ ID NOs: 9 and 10) | | Cethy(57) | R3-H | |
| 7 | IDMTQTPSPVSAAVGDTVTISC(1)- | 4321.1041 | CEthy(22) | R3-L to | Inter |
| | IDISGPYTYYASWAKGR(15) (SEQ ID NOs: 9 | | | R3-H | |
| | and 11) | | | | |
| 8 | QSLEESGGDLVKPGASL(12)-AKGRF(2) | 2385.2295 | Gln→pyro- | R3H to | Intra |
| | (SEQ ID NOs: 12 and 13) | | Glu[AnyN- | (R3-H or | |
| | | | termQ](0) | R1-H) | |
| 9 | IDMTQTPSPVSAAVGDTVTISC(1)- | 3683.7889 | CEthy(22) | R3-L to | Intra |
| | QSSQSVYKNNR(8) (SEQ ID NOs: 9 and 14) | | | R3-L | |
| 10 | IDMTQTPSPVSAAVGDTVTISCQSSQSVYKNNR(30)- | 5667.7254 | Carbamidom | R3L to | Intra |
| | WAFGGGTEVVVKGDPVAPTV(12) (SEQ ID | | ethy[C](22); | (R3-L or | |
| | NOs: 15 and 16) | | Deaminated | R1-L) | |
| | | | [Q](23)[Q]26 | | |
| | | | and [N](32) | | |
| 11 | ISCQSSQSVYKNNR(11)-LAWYQQKPGKPPKL(7) | 3461.7633 | Carbamidom | R3-L to | Intra |
| | (SEQ ID NOS: 17 and 18) | | ethy[C](3) | R3-L | |
| 12 | ISCQSSQSVYKNNRL(11)-AWYQQKPGKPPKL(6) | 3461.7633 | Carbamidom | R3-L to | Intra |
| | (SEQ ID NOs: 19 and 20) | | ethy[C](3) | R3-L | |
| 13 | ISCQSSQSVYKNNRLA(11)-WYQQKPGKPPKL(5) | 3462.7473 | Carbamidom | R3-L to | Intra |
| | (SEQ ID NOs: 21 and 22) | | ethyl[C](3); | R3-L | |
| | | | Deaminated | | |
| | | | [Q](4) | | |
| 14 | ISCQSSQSVYKNNRLAW(11)- | 3687.9314 | Carbamidom | R3-L to | Intra |
| | YQQKPGKPPKLLI(4) (SEQ ID NOs: 23 and 24) | | ethy[C](3) | R3-L | |
| 15 | ISCQSSQSVYKNNRLA(11)-WYQQKPGKPPKL(5) | 3461.7633 | Carbamidom | R3-L to | Intra |
| | (SEQ ID NOs: 21 and 22) | | ethyl[C](3) | R3-L | |
| 16 | ISCQSSQSVYKNNRLAW(11)-YQQKPGKPPKL(4) | 3461.7633 | Carbamidom | R3-L to | Intra |
| | (SEQ ID NOs: 23 and 25) | | ethyl[C](3) | R3-L | |
| 17 | LAWYQQKPGKPPK(7)-QSSQSVYKNNR(8) (SEQ | 2988.5325 | None | R3-L to | Intra |
| | ID NOs: 26 and 14) | | | R3-L | |

The numbers in parentheses correspond to the position of the residues involved in the cross-linking. 1=cross-linking via the amino-terminal end.

### Example 6: Imaged Capillary Isoelectric Focusing (iCIEF) to Separate 4 Antibodies and H/L Pairing Based on PCA Analysis

Four rabbit monoclonal antibodies (rabbit monoclonal mAbs internal naming: R1, P17, P18, P19) were mixed at the same amount (40 μg of each). The sample was desalted using 3 kDa MWCO Amicon® Ultra-0.5 mL centrifugal filter devices (Merck Millipore, Germany) against a buffer containing 20 mM sodium phosphate, pH 7.4.

iCIEF experiment was performed at Advanced Electrophoresis Solutions Ltd (Cambridge, ON, Canada). For preparative iCIEF, 1 mg/ml of the sample in 4% UH 3-10 AESlytes, 6 M urea was mixed with deionised water for a total volume of 40 μl. CEInfinite system with UV detector (280 nm) was used for fraction collection. The focusing was 1 min at 1000 V, 1 min at 2000 V, 10 min at 3000 V, and 3000 V during mobilisation (0.16 μl/min). A total of 6 fractions were collected based on peaks profile controlled and acquired by CEInsight software. The experiment was repeated three more times and each fraction was collected in the same tube.

Sample fractions were reduced, alkylated and deglycosylated using PNGaseF (Promega, WI, US). ProteaseMax Surfactant (Promega, WI, US) was added at a final concentration of 0.01% during trypsin digestion. The digests were loaded onto Evotip Pure™ for mass spectrometry using Whisper method (Orbitrap Exploris 240).

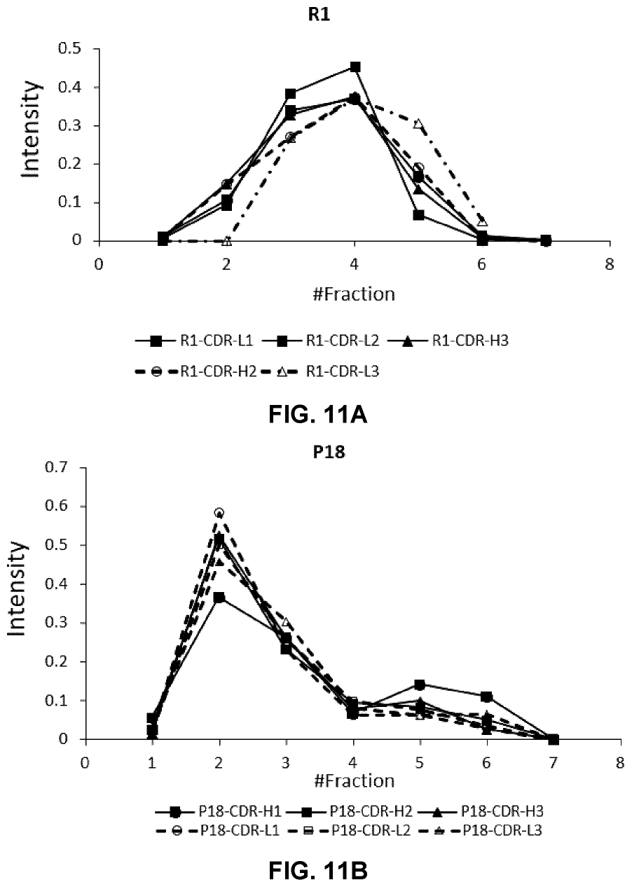

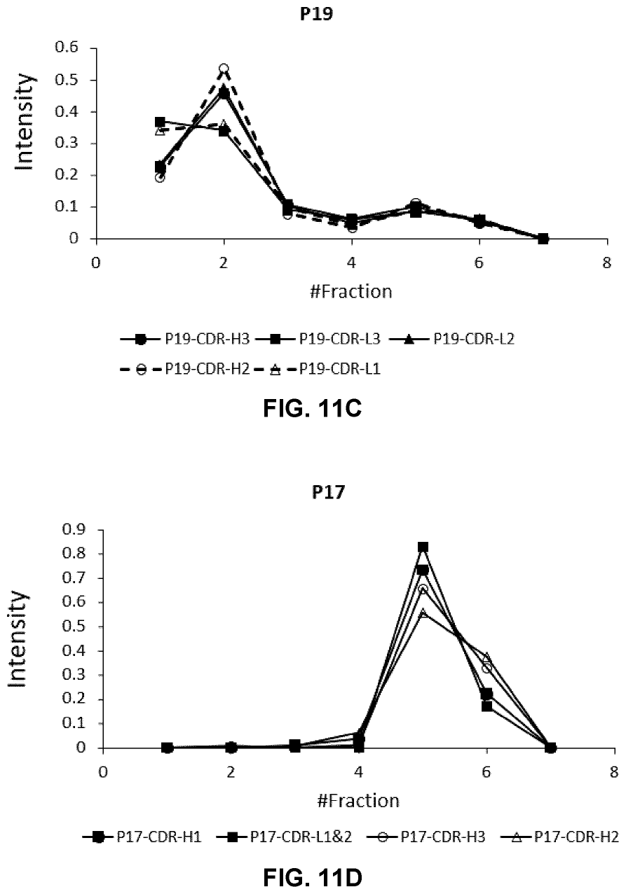

FIG. 10 shows the separation of the mix of 4 rabbit monoclonal and the 6 collected fractions. These fractions were then digested with trypsin and analysed by mass spectrometry on Orbitrap Exploris 240. FIGS. 11A-D show peptide elution profiles across the different fractions. Peptides from heavy and light chain from the 4 different antibodies shows unique peptide elution profiles in which peptides from the same antibody, even from different chains cluster together within the same fraction. This experiment provides evidence that in a mixture of similar antibodies, unique peptides from the heavy and light chains will cluster together, and thus information about the chain pairing can be deduced from the protein elution pattern.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

Fernandez, Pilar (2010). A phase 2, randomized, double-blind safety and pharmacokinetic assessment of respiratory syncytial virus (RSV) prophylaxis with motavizumab and palivizumab administered in the same season. BMC Pediatrics volume 10, Article number: 38 (2010).
Ken Garber. New Discoveries Still Abundant in Monoclonal Antibody Research. Journal of the National Cancer Institute, Volume 92, Issue 18, 20 Sep. 2000, Pages 1462-1464.
Wang X Z, Coljee V W, Maynard J A. Back to the future: recombinant polyclonal antibody therapeutics. Curr Opin Chem Eng. 2013 November; 2(4):405-415.
Bakker AB1, Marissen W E, Kramer R A, Rice A B, Weldon W C, Niezgoda M, Hanlon C A, Thijsse S, Backus H H, de Kruif J, Dietzschold B, Rupprecht C E, Goudsmit J. Novel human monoclonal antibody combination effectively neutralizing natural rabies virus variants and individual in vitro escape mutants. J Virol. 2005 July; 79(14):9062-8.
Cheung, W. C., Beausoleil, S. A., Zhang, X., Sato, S., Schieferl, S. M., Wieler, J. S., Beaudet, J. G., Ramenani, R. K., Popova, L., Comb, M. J. et al. (2012) A proteomics approach for the identification and cloning of monoclonal antibodies from serum. Nature biotechnology, 30, 447-452.
Wine Y, Boutz D R, Lavinder J J, Miklos A E, Hughes R A, Hoi K H, Jung S T, Horton A P, Murrin E M, Ellington A D, Marcotte E M, Georgiou G. Molecular deconvolution of the monoclonal antibodies that comprise the polyclonal serum response. Proc Natl Acad Sci USA. 2013 Feb. 19; 110(8):2993-8. doi: 10.1073/pnas.1213737110. Epub 2013 Feb. 4.
Guthals A, Gan Y, Murray L, Chen Y, Stinson J, Nakamura G, Lill J R, Sandoval W, Bandeira N. De Novo M S/M S Sequencing of Native Human Antibodies. J Proteome Res. 2017 Jan. 6; 16(1):45-54. doi: 10.1021/acs.jproteome.6b00608. Epub 2016 Nov. 2.
Goldstein L. D., Chen, Y J. J., Wu, J. et al. Massively parallel single-cell B-cell receptor sequencing enables rapid discovery of diverse antigen-reactive antibodies. Commun Biol 2, 304 (2019). https://doi.org/10.1038/s42003-019-0551-y).
Fan, S.-B., Meng, J.-M., Lu, S., Zhang, K., Yang, H., Chi, H., Sun, R.-X., Dong, M.-Q. and He, S.-M. 2015. Using pLink to Analyze Cross-Linked Peptides. Curr. Protoc. Bioinform. 49:8.21.1-8.21.19. doi: 10.1002/0471250953.bi0821s49 Marcin Czerwinski, Dorota Siemaszko, Don L. Siegel, and Steven L. Spitalnik The Journal of Immunology, 1998, 160: 4406-4417.
Brandon J DeKosky, Gregory C Ippolito, Ryan P Deschner, Jason J Lavinder, Yariv Wine, Brandon M, Rawlings, Navin Varadarajan, Claudia Giesecke, Thomas Dörner, Sarah F Andrews, Patrick C Wilson, Scott P, Hunicke-Smith, C Grant Willson, Andrew D Ellington & George Georgiou, Nature Biotechnology 31, 166-169(2013)
Jared Shaw, U S 2020/0251318 A1 Direct determination of Antibody chain pairing.
Maibom-Thomsen S L, Trier N H, Holm B E, Hansen K B, Rasmussen M I, Chailyan A, et al. (2019) Immunoglobulin G structure and rheumatoid factor epitopes. PLoS ONE 14(6): e0217624. https://doi.org/10.1371/journal.pone.0217624
WHO link https://www.who.int/rabies/resources/other_rabies_biolog_product/en/
Canadian blood service: https://blood.ca/en/blog/2016-02/wonder-drug-youve-probably-never-heard-yet.
Yu et al., 2016. ECL: an exhaustive search tool for the identification of cross-linked peptides using whole database. BMC Bioinformatics. 2016 May 20; 17(1):217.
Rinner et al., Nat Methods 5(4):315-8, 2008.
Walzthoeni et al., Nat Methods 9(9):901-3, 2012.
Chu et al., Mol Cell Proteomics 9:25-31, 2010.
Trnka et al., Mol Cell Proteomics 2014 February; 13(2):420-34.
Hoopmann et al., J Proteome Res 14(5):2190-198, 2015.
Netz et al. OpenPepXL: An Open-Source Tool for Sensitive Identification of Cross-Linked Peptides in X L-M S. Mol Cell Proteomics. 2020 December; 19(12):2157-2168.
Georg J Pirklbauer, Christian E Stieger, Manuel Matzinger, Stephan Winkler, Karl Mechtler, Viktoria Dorfer. M S Annika: A New Cross-Linking Search Engine. J Proteome Res. 2021 May 7; 20(5):2560-2569.

1. A method for determining heavy and light chain pairing of an antibody or an antibody fragment in an antibody and/or antibody fragment mixture, the method comprising (A): (a) submitting the mixture to a separation step under non-reducing condition to obtain fractions of isolated antibodies or antibody fragments; (b) digesting the isolated antibodies or antibody fragments in the fractions to obtain antibody or antibody fragment peptides; (c) analyzing the antibody or antibody fragment peptides; and (d) determining heavy chain and light chain pairing of the antibody or antibody fragments based on the analysis; and/or (B) (i) contacting the mixture with a cross-linking agent to obtain cross-linked antibodies or antibody fragments: (ii) digesting the cross-linked antibodies or antibody fragments to obtain crosslinked antibody or antibody fragment peptides; (iii) analyzing the crosslinked antibody or antibody fragment peptides; and (iv) determining heavy chain and light chain pairing of the antibody or antibody fragment based on the analysis.

2. (canceled)

3. The method of claim 1, wherein the separation step under non-reducing condition comprises submitting the antibody and/or antibody fragment mixture to a chromatography.

4. The method of claim 3, wherein the chromatography is hydrophobic interaction chromatography (HIC).

5. The method of claim 1, wherein the separation step under non-reducing condition comprises migrating the antibody and/or antibody fragment mixture on a gel.

6. The method of claim 5, wherein the gel is a polyacrylamide or agarose gel.

7. The method of claim 5, wherein the gel is a native gel or a denaturing 2D gel.

8. (canceled)

9. The method of claim 1, wherein the separation step under non-reducing condition comprises submitting the antibody and/or antibody fragment mixture to capillary electrophoresis.

10. The method of claim 9, wherein the capillary electrophoresis is imaged capillary isoelectric focusing (iCIEF).

11. The method of claim 1, further comprises contacting the antibody and/or antibody fragment mixture with an agent that modifies the charge of the protein molecule in a sequence specific manner.

12. The method of claim 11, wherein said agent modifies positively-charged residues into neutral or negatively-charged groups.

13. (canceled)

14. The method of claim 1, wherein the cross-linking agent comprises two N-hydroxysulfosuccinimide (NHS) ester groups.

15. (canceled)

16. The method of claim 1, wherein the method further comprises reducing and alkylating the cysteine residues prior to said digesting.

17. The method of claim 1, wherein the antibody and/or antibody fragment mixture is a polyclonal antibody mixture or a mixture of monoclonal antibodies.

18. The method of claim 1, wherein the digesting comprises contacting the antibodies or antibody fragments with at least one protease.

19. (canceled)

20. The method of claim 1, wherein said analyzing comprises quantifying peptide abundance or intensity of peptide signature from the heavy and light chain across the different fractions, and wherein said determining heavy chain and light chain pairing of the antibody or antibody fragments is based on the amount of peptide from the heavy and light across the different fractions

21. The method of claim 1, further comprising determining the amino acid sequence of at least a portion of the light and heavy chains of the antibodies or antibody fragments present in the mixture.

22. (canceled)

23. The method of claim 1, wherein the step of analyzing the antibody or antibody fragment peptides comprises (A) submitting the antibody or antibody fragment peptides to mass spectrometry (MS) and sequencing the peptides using tandem MS (MS/MS), thereby obtaining MS/MS spectra and/or (B) performing the quantitative profiling of proteotypic peptides unique to each heavy and light chains, and performing pairing based on co-expression of the proteotypic peptides in the same fraction.

24. (canceled)

25. The method of claim 23, wherein the MS/MS spectra are compared either to a database of antibody peptide sequences, or to the sequences of at least a portion of the light and heavy chains of the antibodies or antibody fragments present in the mixture.

26. (canceled)

27. The method of claim 1, wherein the step of analyzing the antibody or antibody fragment peptides comprises performing a clustering analysis.

28. (canceled)

29. The method of claim 1, further comprising expressing a recombinant antibody or antibody fragment comprising the heavy and light chain pair identified.

30-32. (canceled)
